(19) United States
(12) Patent Application Publication
(10) Pub. No.: US 2004/0166071 A1
(43) Pub. Date: Aug. 26, 2004

(54) Rinse-off cosmetic compositions with UV protecting action

(76) Inventors: Matthias Pfaffernoschke, Oberkirch (CH); Maryline Kolly, Givisiez (CH)

Correspondence Address: STRIKER, STRIKER & STENBY, 103 East Neck Road, Huntington, NY, 11743, US

(21) Appl. No.: 10/755,138

(22) Filed: Jan. 9, 2004

(30) Foreign Application Priority Data
Jan. 11, 2003 (DE) 103 00 762.8

(51) Int. Cl. A61K 7/42

(52) U.S. Cl. 424/59

(57) ABSTRACT

Rinse-off cosmetic compositions, which contain a UV-Quat and a UV-filter substance, protect skin and/or hair from damaging UV radiation.

### BACKGROUND OF THE INVENTION

The subject matter of the present invention is a rinse-off, rinse-away or rinse-out cosmetic composition with UV protective action, which contains a combination of a UV-Quat and a UV-filter substance.

It has been known for a long time that ultraviolet light has a damaging effect on unprotected skin and hair. These damaging effects are caused by that UV-portion of sunlight, which is not absorbed by the ozone layer and the oxygen in the air. The UV light filtered out by the earth's atmosphere is in a wavelength range of approximately less than 290 nm and is known as UVC. Damaging effects on the head surface are caused by UVA with a wavelength range between about 400 to 320 nm and UVB with a wavelength range between about 320 to 280 nm, since they can pass through the atmospheric gas layers.

The biological effects of UVA and UVB are many. Besides the desirable reactions in the skin, such as formation of Vitamin D from steroid pre-cursors, UVA and UVB cause a wide spectrum of damage on unprotected regions of the head surface, in which skin and hair are involved. This damage includes especially light to heavy sunburn, erythema, skin necrosis, premature aging, tumors or structure changes in the hair. The production of malignant skin tumors because of repeated exposure to sunlight must be regarded as certain, so that especially advanced steps taken to combat the effects of UVA and UVB radiation must be confirmed or tested. In current times, because of the depletion of the ozone in the ozone layer, protection against UVA and UVB has assumed an every greater importance.

For these reasons the requirements for an effective composition are high. Ideally an effective composition should have a sufficiently high light protective factor (SPF) and a high water resistance. It should contain as small a concentration as possible of the UVB light absorbing material. It should protect against UVA and UVB equally. It must be skin-compatible and hair-compatible and the individual ingredients should be compatible with skin and hair.

Classically hair and skin were protected against UV radiation by use of UV-filters. However they only function when and as long as the UV-filter remains on the skin and/or hair, i.e. essentially only when they are present in a leave-inpreparation.

Thus there is a need to provide an improved and rinsable light protecting composition, in which all the requirements for an effective, long-lasting and skin-friendly and hair-friendly composition are united.

### SUMMARY OF THE INVENTION

It is an object of the present invention to provide a composition for protecting against UV radiation, which has improved properties in comparison to the known means or compositions.

It is a further object of the invention to provide a composition for protecting against UV radiation, which eliminates the disadvantages for the prior art and is formulated as a rinse-off preparation, i.e. an agent, which is designed for rinsing away or off after it is used, but which provides UV protection. Typical rinse-off preparations are agents, such as shampoos, rinses or hair care preparations, which, after application and an acting time of from 1 to 60 minutes, are rinsed again from the hair.

The rinse-off, rinse-away or rinse-out cosmetic composition or preparation for skin and/or hair with UV protective action contains a UV-Quat and a UV-filter substance.

In preferred embodiments the UV-Quat is poly(N-hydroxypolyoxypropylethyl)-N-(adipylpolyoxypropyl)-N-methyl-N-(2-hydroxy-3-cinnamidopropyl dimethylammonium chloride)-methane sulfonate, dodecyl-[3-paradimethylaminobenzamido)-propyl]-dimethylammonium toluene sulfonate or a mixture thereof. Also the UV-filter substance is an organic UVA-filter substance, a UVB filter substance and/or a UVA/UVB filter substance. These latter UV-filter substances are preferably oil-soluble. Preferred embodiments of the UV-filter substance include 4-methoxycinnamic acid-2-ethylhexyl ester and 2-cyano-3, 3-diphenylacrylic acid-2-ethylhexyl ester.

Preferably the weight ratio of the UV-Quat to the UV-filter substance amounts to from 1:2 to 1:3.

The preparation for protecting against UV radiation is preferably a shampoo, a hair conditioning composition or a hair rinse. In preferred embodiments it contains at least one synthetic polymer, preferably an acrylic polymer, especially a cross polymer of an acrylate and an alkyl acrylate or of an acrylate and an allyl ether.

The preparation or composition preferably does not feel oily and contains only about 0.1 to 5 percent by weight or no oily ingredients.

UV-Quats can substantively accentuate negatively loaded hair, since they have quaternized terminal groups, which behave analogously to “normal Quats”. However it was surprisingly found that, due to the combination of at least one UV-Quat with at least one UV-filter substance, there is a significant improvement in the measured DSC peak temperature, which is a measure of hair damage resulting from UV radiation, among other things. Generally the higher the DSC peak temperature, the less the damage to the hair.

DSC (Differential Scanning Calorimetry), according to DIN 51005: 1983-11, also DDK (Dynamic Difference Calorimetry) or DWDK (Dynamic Heat Transport Difference Calorimetry), characterize a method of thermal analysis. Characteristic data (in polymers e.g. glass transition temperature) can be determined in this method by comparing the temperature dependence of the heat transferred to or from a sample and a reference substance.

DSC measurements shown in Table II show the following: that a rinse-conditioner with only a UV-filter substance has a peak temperature of 138.80° C. and a rinse-conditioner with a UV-Quat has a peak temperature of 139.83° C. However the composition according to the invention which has a combination of a UV-filter substance and a UV-Quat, has a peak temperature at 142.95° C., which is almost the same as the peak temperature of untreated hair of 143.75° C.

Thus in fact the protective effect is thus demonstrated, which is based on a synergistic combination of a UV-filter substances and a UV-Quat. This may also be shown by tearing force measurements on irradiated hair.

It was also unexpectedly found that the protection of the hair from UV can be substantially improved by the synergistic combination of a UV-filter substance and a UV-Quat, especially in cationic rinse-off products. For this purpose it is unimportant whether UV-light absorbing inorganic pigments are contained in the composition as UV-filter substance or whether the organic UV-filter substance contained in it are water-soluble or oil-soluble. The explanation for this surprising effect is apparently that the UV-filter substance is not or only slightly rinsed away, when it is used in combination with a UV-Quat. To obtain this effect a weight excess of the UV-filter substance in comparison to the UV-Quat suffices.

All commercially available UV-Quats, which have at least one quaternary ammonium group and at least one UV-light absorbing group, can be considered as the UV-Quat. The UV-Quats absorb UV radiation in the following wavelength ranges: UV-A, 400 to 320 nm; UV-B, 320 to 280 nm; UV-C, 280 to 200 nm. Suitable UV-Quats are, for example, described in U.S. Pat. No. 4,061,730 A and U.S. Pat. No. 5,427,773 A.

Suitable UV-Quats are:

Poly(N-hydroxypolyoxy-propylethyl)-N-(adipylpolyoxypropyl)-N-methyl-N(2-hydroxy-3-cinnamidopropyl-dimethylammonium chloride)-methane sulfonate (INCI Name:Polyquaternium-59 (and) Butylene Glycol), marketed by Croda Inc., Parsippany, N.J. 07054, USA; under the trademark, CRODASORB® UV-HPP; and

Dodecyl-[3-paradimethyl-aminobenzamido)-propyl]-dimethyl ammonium toluene sulfonate/Propan-1,2-diol-1-octadecanoate (INCI Name: Dimethyl PABAmidopropyl Laurdimonium Tosylate (and) Water (and) Propylene Glycol Stearate), marketed by ISP Chemicals NJ 07470, USA, under the trademark ESCALOL® HP 610.

In contrast to UV-Quats, the UV-filter substance is understood to be a UV-light absorbing inorganic or organic filter substance without quaternary nitrogen, which can be used in the cosmetic composition.

All micropigments or pigments known for this application can be considered as the UV-light absorbing inorganic pigment, especially all water-insoluble or slightly soluble metal or metalloid compounds in ionic, non-ionic or oxidized form. These pigments can be present in this form individually or as mixtures or individually or as mixtures of mixed oxides, in which the mixtures of mixed oxides include mixtures with pure oxides. For example titanium oxides (e.g. TiO₂), zinc oxides (e.g. ZnO), aluminum oxides (e.g. Al₂O₃), iron oxides (e.g. Fe₂O₃), manganese oxides (e.g.MnO), silicon oxides (e.g. SiO₂), silicates, cerium oxides, zirconium oxides (e.g. ZrO₂), barium sulfates (BaSO₄), or mixtures of the foregoing should be mentioned. Suitable pigments and/or micropigments are obtained commercially, for example, HOMBITEC® L5 (INCI Name:Titanium dioxide) of Merck.

UV-light absorbing inorganic pigments can be contained in the composition according to the invention in a total amount between 0.01 to 20.0 percent by weight, especially between 0.05 to 10.0 percent by weight, preferably between 0.05 to 5.0 percent by weight in relation to the total amount of the composition.

All known UVA-, UVB- and UVA/UVB-filter substances, individually or in combination with each other, which can be contained in cosmetic and/or dermatological preparations, can be used as the organic UV-filter substances. The UV-filter substances can be water-soluble or oil-soluble. In preferred embodiments of the composition according to the invention the pigment is worked into an aqueous phase and the organic UV-filter substance is worked into an oil phase, oil-soluble UV-filter substances are preferred in these embodiments.

For example, derivatives of dibenzoylmethanes (e.g. Parsol 1789 of Givaudan/Roure, INCI name: butyl methoxydibenzoyl methane) can be used as is the UVA-filter substance.

Any of the following compounds can be used as the UVB-filter substance:

Benzylidene camphor or derivatives thereof, especially methylbenzylidene camphor (e.g. 3-benzylidene camphor, 3-(4-methylene benzyliden)-dl-camphor), derivatives and esters of cinnamic acids, especially derivatives and esters of methoxycinnamic acid (e.g. 4-methoxycinnamic acid octyl ester or 4-methoxycinnamic acid isopentyl ester, 4-methoxycinnamic acid 2-ethylhexyl ester (INCI: ethylhexyl methoxycinnamate)), derivatives and esters of benzoic acid, especially 4-aminobenzoic acid; 2-cyano-3,3-diphenylacrylic acid-2-ethylhexyl ester (INCI: octocrylene), polyhydroxybenzoic acid (e.g. polyhydroxybenzoic acid methyl ester or polyhydroxybenzoic acid propyl ester); esters of salicylic acid (for example silicylic acid (2-ethylhexyl)ester or salicylic acid (4-isopropylbenzyl)ester).

As water-soluble UV-filter substances for example the following should be mentioned: sulfonic acids, benzophenones and their derivatives, for example the sulfonic acid derivatives of benzophenone (for example 2-hydroxy-4-methoxybenzophenon-5-sulfonic acid) as UVB/UVA-filter substances or the benzimidazoles (for example 2-phenylbenzimidazol-5-sulfonic acid) and their salts, especially the sodium and potassium salts.

Additional UV-filter substances include: dibenzoylmethane or suitable polypeptides, especially oxygen radical trapping agents, for example the known Mn-, Fe- or Zn-superoxide desmutases, as well as tocopherols and Vitamins (e.g. ascorbic acid).

The organic UV-filter substances, 4-methoxycinnamic acid-2-ethylhexyl ester and 2-cyano-3,3-diphenylacrylic acid-2-ethylhexyl ester, are particularly preferred.

The organic UVA-, UVB- or UVB/UVA-filter substances can be contained in the composition according to the invention in a total amount of between 0.1 to 30.0 percent by weight, especially between 0.5 to 25.0 percent by weight, preferably between 0.3 and 5.0 percent by weight, in relation to the total amount of the composition.

The weight ratio of UV-Quat to UV-filter substance should preferably be in a range of 1:1 to 1:15, especially preferably from 1:1 to 1:5 and most preferably from 1:2 to 1:3.

Preferred embodiments of the composition according to the invention can contain a synthetic polymer. The synthetic polyer is preferably an acrylic polymer, especially a cross polymer of an acrylate and alkyl acrylate and/or an acrylate and an allyl ether. For example PEMULEN®-, CARBOPOL®- and ACRISINT® Type, for example PERMULEN® TR1 of Goodrich, (INCI Name: Acrylates/C10-30 Alkyl Acrylate Crosspolymer); CARBOPOL® 1382 of Goodrich (INCI Name: Acrylates/C10-30 Alkyl Acrylates Crosspolymer); CARBOPOL® 2984 of Goodrich (INCI Name: Carbomer); or ACRISINT® 400 of 3 V (INCI Name: Carbomer), which can be present individually or in combination in the composition according to the invention. The concerned synthetic polymers can be worked into the oil phase or lipid phase or into the water phase, preferably into the water phase, of the composition according to the invention. They are contained in a total amount of between 0.05 and 5.0 percent by weight, especially between 0.1 and 3.0 percent by weight, especially preferably between 0.1 and 1.0 percent by weight, in relation to the total amount of the composition.

Understandably the composition designed for rinsing after application can contain all those conventional and known additive ingredients, e.g. thickeners, such as bentonite, fatty acids, starches, polyacrylic acids and their derivatives, cellulose derivatives, alginates, VASELINE®, paraffin oils; wetting agents or emulsifiers from the classes of anionic, cationic, amphoteric and nonionic surface-active substances, e.g. fatty alcohol sulfates, fatty alcohol ether sulfates, alkyl sulfonates, alkyl benzene sulfates, fatty alcohol ether sulfates, alkyl sulfonates, alkyl benzene sulfates, quaternary ammonium salts, alkyl betaines, ethoxylated alkyl phenols, fatty acid alkanol amides or ethoxylated fatty acid esters; turbidity-inducing agents, such as polyethylene glycol esters; alcohol, such as ethanol, propanol, isopropanol, and polyols, such as ethylene glycol, 1,2 or 1,3-propan-diol, 1,2-, 1,3- or 1,4-butandiol; 1,2-, 1,3-, 1,4- or 1,5-pentandiol and glycerol. An ethylene glycol ether, especially an ethylene glycol monophenyl ether, is suitable as an ethylene glycol monophenyl ether additive in the composition of the invention. For example phenoxetol of Nipa (INCI name: phenoxyethanol) is suitable. Additional suitable additive ingredients include sugars, such as D-glucose, solvating agents, stabilizers, buffer substances, perfume oils, dye substances and hair conditioning and hair care ingredients, such as cationic polymers, lanolin derivative compounds, cholesterol, pantothenic acid and betaine.

The conventional cosmetic additive ingredients are used in the composition according to the invention in amounts suitable for their purposes, e.g. the wetting agents and emulsifiers are used in total amounts of 0.2 to 30 percent by weight; the alcohol in amounts of 0.5 to 30 percent by weight, especially of 1 to 25 percent by weight, preferably of 2 to 20 percent by weight, most preferably of 3 to 15 percent by weight, in relation to the total amount of the composition; the turbidity-inducing agents, perfume oils and dye compositions, in an amount of about 0.01 to 1 percent by weight, respectively; the buffer substances in an amount of 0.1 to 10 percent by weight; the sugars, solvating agents, stabilizers and hair conditioning and hair care ingredients in an amount of 0.1 to 10 percent by weight each, but the total amount of the thickeners and the solvating agents is from 0.5 to 20 percent by weight. Aromatic ether alcohol can be present in the composition of the invention in an amount between 0.1 to 10 percent by weight, especially between 0.2 and 5 percent by weight, especially preferably between 0.5 to 2 percent by weight.

Understandably the composition according to the invention can contain all further additive, auxiliary and carrier substances suitable for skin and hair cosmetics.

Basically one skilled in the art knows which additive, auxiliary and carrier substances are used in hair and skin cosmetics. Thus the next following embodiments are only exemplary and serve only for further illustration of the present invention.

For this purpose additional reference is made to the available literature, which describes the general formulation of such compositions, for example K. Schrader, Foundation and Formulation of Cosmetics [Grundlagen und Rezepturen der Kosmetika], 2^{nd} Edition, 1989; or A. DOMSCH, Cosmetic Preparations [Die kosmetischen Präparate], Chemical Industry Press (H. Ziolkowsky, Ed.), 4^{th} Edition, 1992; or N. J. Lowe & N. A. Shaat, Sunscreens Development Evaluation and Regulatory Aspects, Marcel Dekker Inc., 1990.

For example, only a few of the further additive, auxiliary and carrier substances will be mentioned here; this enumeration will be left unfinished. Among the thickeners for example, clay, starch, polyacrylic acids and their derivatives, cellulose derivatives or alginates can be mentioned. Additional hair and skin care substances include, for example, sugars, proteins, lanolin derivatives, vitamins or provitamins, for example biotin, Vitamin C or Dpanthenol. The further additive, auxiliary and carrier substances include defatting agents, inorganic acids, organic acids (for example lactic acids, citric acids, glycolic acids, phosphoric acids); preservative agents (for example parahydroxybenzoic acid ester), nonaqueous solvents, antioxidants (such as tocopherol and esters of tocopherol), dyestuffs and perfumes or fragrances.

The further additive, auxiliary and carrier substances can be used in the usual amounts known by those skilled in the art and are worked into according to the known methods.

The composition according to the invention can be present in different administration forms, which are known for cosmetic skin and hair products for protection from sunlight. For example they can be in the form of shampoos, rinses, gels, creamy gels, creams, lotions, shakable mixtures, sprays or foams.

A conditioning shampoo, a conditioning rinse or a rinse-off hair care composition is preferred for the cosmetic composition for protecting skin and/or hair from UV radiation. However the cosmetic composition can be predominantly designed to protect the hair or the skin from UV radiation, such as a sunscreen. Also it can be rinsed off again after application and action. However the UV-protection still remains after that.

The composition according to the invention can be made in the usual manner. The addition and mixture of conventional auxiliary and carrier substances can occur according to the known methods.

As organic UV-filter substances also water-soluble UV-filter substances can be selected, which can be worked into the water and/or lipid phase either individually or together with an oil-soluble UV-filter substance.

Consequently a method for making the cosmetic composition according to the invention is also possible in which the organic UV-filter substance is water-soluble and/or oil-soluble and is worked into the water and/or oil or lipid phase.

Preferably the composition according to the invention however contains no oily and/or fatty substances or only in a small amount of from 0.1 to 5 percent by weight, in relation to the total amount of the composition. In addition, preferably the composition according to the invention does not feel oily or fatty.

In an additional method for making the composition according to the invention the synthetic polymer can be added. In this additional method the synthetic polymer is preferably worked into the aqueous phase. For this purpose acrylic polymers are preferred, especially a cross polymer of an acrylate and an alkyl acrylate or of an acrylate and an allyl ether.

### EXAMPLES

The following examples illustrate the present invention in greater detail. The amounts of the ingredients are given in percentages by weight and are related to the total amount or total weight of the composition. The individual ingredients and raw materials are commercially obtainable and have the following INCI names. Reference is made to the known literature, for example the International Cosmetic Ingredient Dictionary and Handbook Seventh Edition, 1997:

TABLE I
INGREDIENT NAMES AND MANUFACTURERS

| RAW MATERIAL/ | | |
|---|---|---|
| COMMERCIAL | | MANU- |
| NAME | INCI NAME | FACTURER |
| ABIL WAX ® | Cetyl Dimethicone | Goldschmidt |
| 9801D | | |
| ACRISINT ® 400 | Carbomer | 3V |
| AMPHISOL ® K | Potassium Cetyl Phosphate | Givaudan/Roure |
| ANTARON ® | PVP/Hexadecene | ISP |
| V-216 | Copolymer | |
| ANTARON ® | PVP/Eicosene Copolymer | ISP |
| V-220 | | |
| BETAIN | Betaine | Amino |
| MONOHYDRATE | | |
| CARBOPOL ® | Acrylates/C10-30 Alkyl | Goodrich |
| 1382 | Acrylate Crosspolymer | |
| CARBOPOL ® | Carbomer | Goodrich |
| 2984 | | |
| CARBOPOL ® | Carbomer | Goodrich |
| Ultrez 10 | | |
| CETIOL ® 868 | Octyl Stearate | Cognis |
| CETIOL ® SN | Cetearyl Isononanoate | Cognis |
| COLORONA ® | Mica (and) CI77891 (and) | Merck |
| ORIENTAL | CI 77491 | |
| BEIGE 17237 | | |
| Crodasorb UV-HPP | Polyquaternium-59 | Croda |
| DEKABEN ® LMB | Iodopropynl Butyl | Jan Dekker |
| D-Panthenol | carbamate | BASF |
| | Panthenol | |
| DRAGOSANTOL ® | Bisabolol | Dragoco |
| EDETA ® BD | Disodium EDTA | BASF |
| Empicol ESB 3/WF | Sodium Laureth Sulfate | Huntsman |
| | | Surface Science |
| EUSOLEX ® 6300 | 4-methylbenzylidene | Merck |
| | camphor | |
| EUTANOL ® G | Octyldodecanol | Cognis |
| ESCALOL ® HP610 | Dimethyl PABAmidopropyl | Van Dyk |
| | Laurdimonium Tosylate | |
| ESCALOL ® 507 | Octyl Dimethyl PABA | Van Dyk |
| EXTRAPON ® | Witch Hazel Distillate | Dragoco |
| Hamamelis | | |
| FINSOLV ® TN | C12-15 Alkyl Benzoate | Finetex |
| GENAMIN CTAC 50 | Cetrimonium Chloride | Clariant |
| HOMBITEC ® I5 | Titanium Dioxide | Merck |
| HYDROLITE ®- | Pentylene Glycol | Dragoco |
| 5 2/016020 | | |
| LANETTE ® O | Cetearyl Alcohol | Cognis |
| NEO HELIOPAN ® | Ethylhexyl Methoxy- | Haarmann & |
| AV/OA | cinnamate | Reimer |
| NEO HELIOPAN ® | Octocrylene | Haarmann & |
| Type 303 | | Reimer |
| PARSOL ® 1789 | Butyl Methoxydibenzoyl- | Givaudan/Roure |
| | methane | |
| PHENOXETOL ® | Phenoxyethanol | Nipa Laborartories |
| PEMULEN ® TR1 | Acrylates/C10-30 Alkyl | Goodrich |
| | Acrylate Crosspolymer | |
| Salcare SC 92 | Polyquaternium-32 | Ciba |
| TANNOGEN ® | Hydrolyzed Soy Protein | Laboratoires |
| LS 1463 C | | Serobiologiques |
| TEGO BETAIN | Cocamidopropyl Betaine | Goldschmidt |
| L 5045 | | |
| Vitamin E Acetate | Tocopheryl Acetate | BASF |
| PHB Methyl ester | Methyl parabene | Chemag |
| PHB Propyl ester | Propyl parabene | Chemag |
| Ucare Polymer JR 400 | Polyquaternium-10 | Amerchol |

### Example 1

### Conditioner with UV-Filter

| | | |
|---|---|---|
| LANETTE ® O | 3.00 | g |
| Genamin CTAC 50 | 1.00 | g |
| NEO HELIOPAN ® AV/OA | 2.00 | g |
| NEO HELIOPAN ® Type 303 | 0.50 | g |
| Perfume | 0.30 | g |
| D-Panthenol | 0.50 | g |
| Water, desalinized | to 100.00 | g |

### Example 2

### Conditioner with UV-Quat (State of the Art)

| | | |
|---|---|---|
| LANETTE ® O | 3.00 | g |
| Genamin CTAC 50 | 1.00 | g |
| ESCALOL ® HP 610 | 2.00 | g |
| Perfume | 0.30 | g |
| D-Panthenol | 0.50 | g |
| Water, desalinized | to 100.00 | g |

### Example 3

### Conditioner with UV-QUAT AND UV-Filter

| | | |
|---|---|---|
| LANETTE ® O | 3.00 | g |
| Genamin CTAC 50 | 1.00 | g |
| ESCALOL ® HP 610 | 2.00 | g |
| NEO HELIOPAN ® AV/OA | 2.00 | g |
| NEO HELIOPAN ® Type 303 | 0.50 | g |
| Perfume | 0.30 | g |
| D-Panthenol | 0.50 | g |
| Water, desalinized | to 100.00 | g |

### Example 4

### Shampoo with UV-QUAT AND UV-Filter

| | | |
|---|---|---|
| Empicol ESB 3/WF | 30.00 | g |
| TEGO BETAIN L 5045 | 7.50 | g |
| Ucare Polymer JR 400 | 0.50 | g |
| NEO HELIOPAN ® AV/OA | 2.00 | g |
| Crodasorb UV-HPP | 1.50 | g |
| Perfume | 0.30 | g |
| D-Panthenol | 0.50 | g |
| Water, desalinized | to 100.00 | g |

The UV protective action of compositions of the prior art and the UV protective action of a composition according to the invention with the synergistic action due to the combination of the UV-Quat and the UV-filter substance are reported in the following. The measurements of DSC peak temperatures, according to the following Table II for rinse-conditioners, serve for that purpose.

TABLE II
DSC Peak Temperatures of Different Rinse-Conditioners

| | DSC-Peak |
|---|---|
| Test Sample | Temperature, ° C. |
| Reference untreated, unradiated | 143.75 |
| Reference untreated, radiated | 139.93 |
| Rinse-Condtiioner according to Example 1 with | 138.80 |
| UV-filter (Prior Art) | |
| Rinse-Condtiioner according to Example 2 with | 139.83 |
| UV-Quat (Prior Art) | |
| Rinse-Condtiioner according to Example 3 with | 142.95 |
| UV-Quat and UV-filter (Invention) | |

As shown in Table II, the combination of the UV-Quat and UV-filter substance in the rinse-off conditioners leads to a definite increase of the DSC peak temperatures, which shows a surprising definite increase of UV-protective action, which produces almost the same value as for hair that is not radiated and thus is considerably better than the protection, which may be provided with only the UV-Quat or only the UV-filter.

The disclosure in German Patent Application 103 00 762.8 of Jan. 11, 2003 is incorporated here by reference. This German Patent Application describes the invention described hereinabove and claimed in the claims appended hereinbelow and provides the basis for a claim of priority for the instant invention under 35 U.S.C. 119.

While the invention has been illustrated and described as embodied in a rinse-off or rinse-away cosmetic composition with UV protecting action, it is not intended to be limited to the details shown, since various modifications and changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed is new and is set forth in the following appended claims.

We claim:

1. A rinse-off or rinse-away cosmetic composition for protecting skin and/or hair from UV radiation, said composition containing a UV-Quat and a UV-filter substance.

2. The composition as defined in claim 1, wherein said UV-Quat is selected from the group consisting of poly(N-hydroxypolyoxy-propylethyl)-N-(adipylpolyoxypropyl)-N-methyl-N-(2-hydroxy-3-cinnamidopropyl-dimethylammonium chloride)-methane sulfonate and dodecyl-[3-paradimethyl-aminobenzamido)-propyl]-dimethylammonium toluene sulfonate.

3. The composition as defined in claim 1, wherein said UV-filter substance is selected from the group consisting of UVA-filter substances, UVB-filter substances and UVA/UVB-filter substances.

4. The composition as defined in claim 3, wherein said UV-filter substance is oil-soluble.

5. The composition as defined in claim 1, wherein said UV-filter substance is selected from the group consisting of 4-methoxycinnamic acid-2-ethylhexyl ester and 2-cyano-3,3-diphenylacrylic acid-2-ethylhexyl ester.

6. The composition as defined in claim 1, wherein said UV-Quat and said UV-filter substance are present in a weight ratio of said UV-Quat to said UV-filter substance of from 1:2 to 1:3.

7. The composition as defined in claim 1, consisting of a shampoo or a hair rinse.

8. The composition as defined in claim 1, further comprising at least one synthetic polymer.

9. The composition as defined in claim 8, wherein said at least one synthetic polymer is an acrylic polymer.

10. The composition as defined in claim 9, wherein said acrylic polymer is a cross polymer of an acrylate and an alkyl acrylate or a cross polymer of an acrylate and an allyl ethers.

11. The composition as defined in claim 1, consisting of a hair-conditioning rinse or a hair care package.

12. The composition as defined in claim 1, containing from 0.1 to 5 percent by weight of oily ingredients and not feeling oily.

13. The composition as defined in claim 1, containing no oily ingredients and not feeling oily.

14. A method of protecting skin and/or hair from UV radiation, said method comprising the steps of:
a) providing a rinse-off cosmetic composition containing a UV-Quat and a UV-filter substance;
b) applying said rinse-off cosmetic composition to said skin and/or hair and allowing said cosmetic composition to act on the hair; and
c) rinsing way or rinsing off said cosmetic composition after said applying and said allowing of step b).
